(19) United States
(12) Patent Application Publication
(10) Pub. No.: US 2021/0205001 A1
(43) Pub. Date: Jul. 8, 2021

(54) SURGICAL SHAVING INSTRUMENTS

(71) Applicant: GYRUS MEDICAL LIMITED, Cardiff (GB)

(72) Inventor: Ole SCHLOTTMANN, Cardiff (GB)

(73) Assignee: GYRUS MEDICAL LIMITED, Cardiff (GB)

(21) Appl. No.: 17/138,114

(22) Filed: Dec. 30, 2020

(30) Foreign Application Priority Data
Jan. 7, 2020 (GB) 2000165.7

(51) Int. Cl. A61B 18/14; A61B 17/32

(52) U.S. Cl. CPC A61B 18/148; A61B 2018/00577; A61B 17/32002; A61B 18/1445

(57) ABSTRACT

An electrosurgical instrument that includes a hollow elongate shaft having a first axis and extending from a proximal end region to a distal end region. The instrument includes a tip portion at the distal end region that has a cavity. The cavity includes a base surface and a first retention surface extending away from the base surface in a direction towards the distal end region. The instrument also includes a conductor extending within the shaft and an electrode in the cavity and in electrical communication with the conductor. The electrode includes a first electrode element and a second electrode element in electrical connection and located adjacent one another. The second electrode element abuts and extends adjacent the first retention surface to restrain the movement of the electrode in a direction towards the outer surface of the tip portion and the proximal end region of the shaft.

The present invention relates to electrosurgical instruments, and in particular arthroscopic electrosurgical instruments.

### BACKGROUND OF THE INVENTION

Electrosurgical instruments are used by surgeons to perform surgical resection operations. One example of such an instrument is a suction electrode, which is used for the resection, ablation and excision of soft tissue as well as the coagulation and haemostasis of blood vessels during arthroscopic procedures.

Such instructions comprise an elongate shaft with a tip portion provided at the distal end thereof. The distal tip portion is typically provided with electrically conductive electrodes for supplying coagulating and ablating and ablation radio frequency (RF) energy to the targeted tissue. Typically, the elongate shaft and distal tip are of a metallic material and, in the case of electrodes provided at the distal tip, a thin layer of insulation is provided in order to insulate the electrically conductive electrode from the remainder of the distal end thereof.

Conventional distal tip retention on the elongate shaft involves the distal tip extending through the ceramic insulator and being held in place by means of additional riveting or weld plates or the other mechanical attachment means. Secondary retention is also often provided using adhesive and/or active wire connection. However, all of these distal tip retention means are relatively bulky and result in a distal tip with a significant profile.

However, as the instrument is used in arthroscopic surgery (often referred to as keyhole surgery), surgery is performed using an arthroscope i.e. an endoscope that is inserted into the joint through a small incision. Therefore, the size of the device is an important consideration. The lower the profile of the distal tip the better, both for facilitating surgery via a small incision and also from a perspective of accuracy of delivery of radiofrequency through the electrically conductive electrodes at the distal tip of the instrument.

There is therefore a need for an electrosurgical instrument having a retained low-profile distal tip.

The present invention seeks to address the issues of the prior art.

### SUMMARY OF THE INVENTION

Accordingly, a first aspect of the present invention provides an electrosurgical instrument comprising: a hollow elongate shaft extending from the main body along a first axis, the shaft defining an elongate inner open volume which extends from a proximal end region to a distal end region of the shaft; a tip portion extending from the distal end region of the elongate shaft, the tip portion comprising an electrically insulating material and having an outer surface, the tip portion further defining a cavity therein, the cavity comprising a base surface and a first retention surface extending away from the base surface in a direction towards the distal end region of the shaft at a non-transverse angle to the first axis; a conductor extending within the shaft from the proximal end region to the distal end region, wherein the proximal end region is suitable for connection to a source of electrosurgical energy; and an electrode received within the cavity and in electrical communication with the conductor, the electrode comprising a first electrode element and a second electrode element in electrical connection with the first electrode element, wherein the first and second electrode elements are located adjacent one another and the second electrode element abuts and extends along the first retention surface to restrain the movement of the electrode in a direction towards the outer surface of the tip portion and in a direction towards the proximal end region of the shaft.

By use of a first retention surface that extends at a non-transverse angle i.e. an angle that is not at 90° to the first axis, the first retention surface acts to restrain the movement of another abutting body away from the first axis in a direction transverse to the first axis. Thus, the electrode is restrained from movement towards the outer surface of the tip portion by abutment with the first retention surface of the cavity.

In one embodiment, the cavity further comprises a second retention surface extending away from the base surface towards the outer surface at an angle transverse to the first axis, the second retention surface facing towards the proximal end region, and wherein the first electrode element abuts the second retention surface and restrains movement of the electrode in a direction towards the distal end region of the shaft.

As the second retention surface extends at an angle transverse to the first axis and faces in the proximal direction, the second retention surface acts to restrain the movement of another abutting body away from the first axis in the distal direction. Thus, the electrode is restrained from movement in the distal direction by abutment with the second retention surface of the cavity.

In a further embodiment, the cavity further comprises a third retention surface extending away from the base surface towards the outer surface at an angle transverse to the first axis, the third retention surface facing towards the distal end region, and wherein the first and second electrode elements abut the third retention surface and restrain movement of the electrode in a direction towards the proximal end region of the shaft.

As the third retention surface extends at an angle transverse to the first axis and faces in the distal direction, the third retention surface acts to restrain the movement of another abutting body away from the first axis in the proximal direction. Thus, the electrode is restrained from movement in the proximal direction by abutment with the third retention surface of the cavity.

In a further embodiment, the conductor extends into the cavity and engages the first and second electrode elements. Preferably, the first and second electrode elements define an aperture dimensioned to receive a portion of the conductor therein and wherein the conductor is located within the aperture to restrain movement of the electrode in a direction transverse to the first axis.

The conductor extends within the shaft of the electrosurgical instrument. Thus, the engagement of the conductor in an aperture defined between the first and second electrode elements serves to anchor the electrode and restrain movement of the electrode in any direction transverse to the first axis.

In a further embodiment, the tip portion cavity comprises a continuous channel extending around the periphery of the tip portion and surrounding the outer surface. Thus, the electrode is located within the channel and arranged around the outer surface.

Preferably, the first and second electrode elements are welded together within the cavity. In this way, there is no requirement for additional components to secure the tip portion and electrode in place and thus a lower profile tip portion can be attained.

The electrosurgical instrument of the present invention is preferably further provided with a return brace defining an aperture corresponding to the outer surface, wherein the return brace is located adjacent the tip portion and the second electrode element, with the aperture aligned with the outer surface of the tip portion.

The electrosurgical instrument of the present invention may comprise, but is not restricted to, a bone bur, suction coagulator, bipolar forceps and the like.

### BRIEF DESCRIPTION OF THE DRAWINGS

FIG. 1 is a perspective view from one side of an embodiment of an electrosurgical instrument in accordance with a first aspect of the present invention;

FIG. 2 is a perspective view from one side and above of the tip portion of the embodiment of FIG. 1;

FIG. 3 is a perspective view from above of the tip portion of FIG. 2;

FIG. 4 is a cross-sectional view of the embodiment of the tip portion of FIG. 2; and

FIG. 5 is a an exploded view of the embodiment of FIG. 1.

### DETAILED DESCRIPTION OF THE PREFERRED EMBODIMENTS

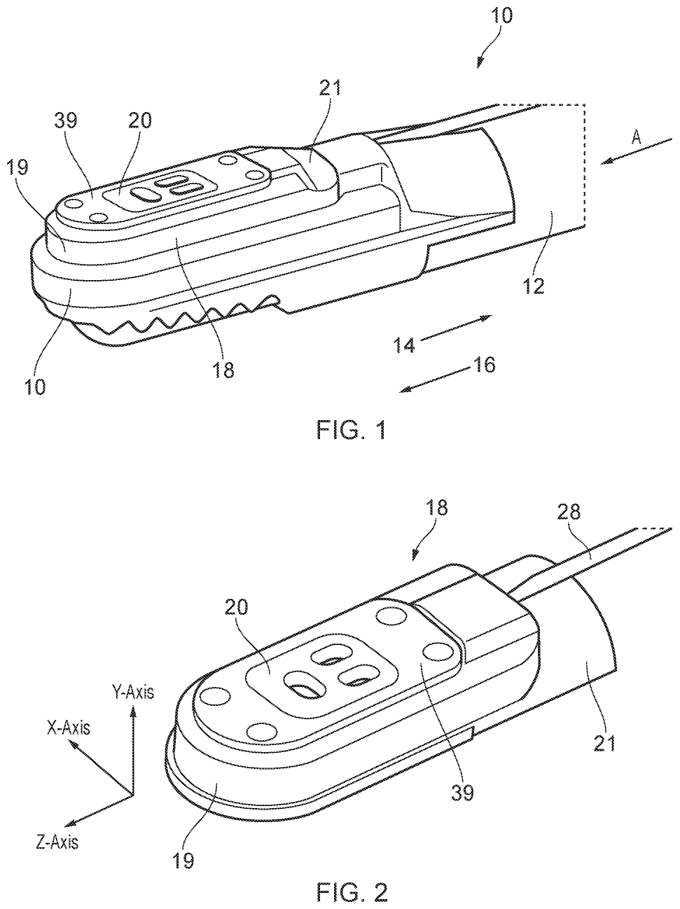

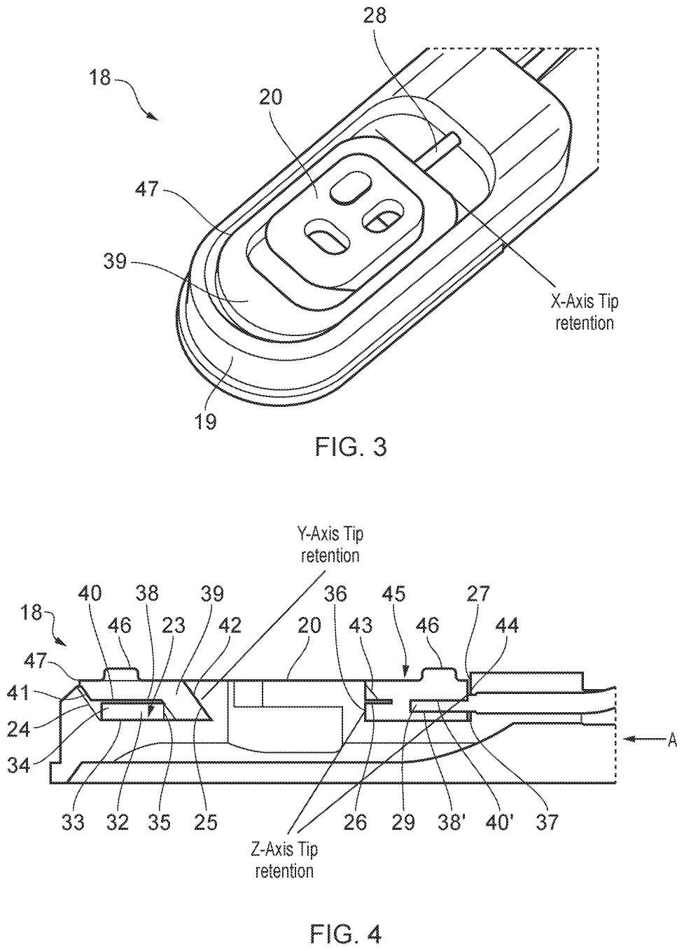

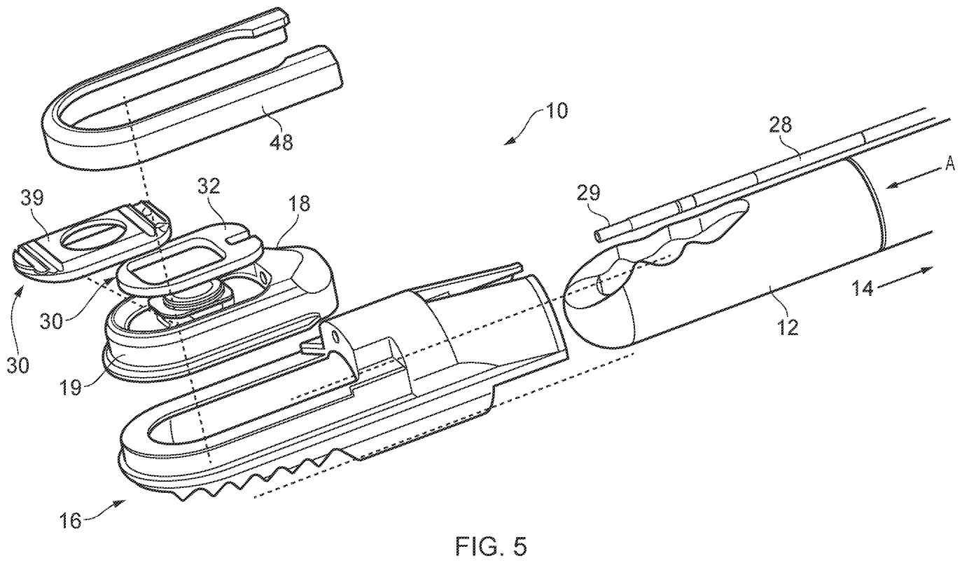

FIG. 1 shows an electrosurgical instrument 10 comprising an elongate shaft 12 having a proximal end region 14 and a distal end region 16. Elongate shaft 12 is hollow and defines an elongate open volume which extends along the length of shaft 12. Elongate shaft 12 defines a first axis A extending therethrough.

Electrosurgical instrument 10 further comprises a tip portion 18 extending from the distal end region 16 of elongate shaft 12. Tip portion 18 defines a distal portion 19 and a proximal portion 21 and comprises an electrically insulating material such as, but not restricted to, ceramic. Tip portion comprises an outer surface 20 and defines a cavity 22 therein. Cavity 22 has a base surface 23 extending in a plane parallel to the first axis A and first and second distal surfaces 24, 25 extending from base surface 23. First and second proximal surfaces 26, 27 also extend from base surface 23.

First and second proximal surfaces 26, 27 extend from base surface 23 at an angle transverse to axis A.

Second proximal surface 27 defines an aperture 27′ therein dimensioned to receive a portion of electrical conductor 28 therethrough.

First distal surface 24 extends away from base surface 23 of cavity 22 at an obtuse angle (x) relative to base surface 23, in a direction towards distal portion 19 of tip portion 18. Second distal surface 25 extends away from base surface 23 of cavity 22 at an acute angle (y) relative to base surface 23 in a direction towards distal portion 19 of tip portion 18. In the embodiment shown in FIG. 4, the first and second distal surfaces are substantially parallel to one another.

An electrical conductor 28 extends within shaft 12 from the proximal end region 14 to the distal end region 16. Electrical conductor 28 is connectable to a source of electrosurgical energy (not shown) such as microwaves or RF waves.

Electrosurgical instrument 10 further comprises an electrically conductive electrode 30 received within cavity 22 of tip portion 18. Electrode 30 comprises a first electrode element 32, and a second electrode element 39 in electrical connection with first electrode element 32. Both first and second electrodes 32, 39 are welded together after location within the cavity 22 to form one electrode.

First electrode element 32 and second electrode element 34 are located adjacent one another with second electrode element 34 being exposed at the surface of tip portion 18. First electrode element 32 is retained below second electrode element 34 and is not exposed at the surface of tip portion 18.

First electrode element 32 comprises a base surface 33 and upper surface 38, and first and second distal surfaces 34, 35, and first and second proximal surfaces 36, 37, all extending from base surface 33. All of first and second distal surfaces 34, 35 and first and second proximal surfaces 36, 37 extend away from base surface 33 in a direction transverse to base surface 33. A portion of upper surface 38 defines a channel 38′ with a cross-sectional contour dimensioned to receive a portion of the distal portion 29 of electrical conductor 28 therein.

Second electrode element 39 comprises a base surface 40, and first and second distal surfaces 41, 42, and first and second proximal surfaces 43, 44, all extending from base surface 40. First distal surface 41 extends away from base surface 40 at an obtuse angle (x) as shown in FIG. 4 such that, when located within cavity 22, first distal surface 41 extends away from base surface 40 at the same obtuse angle (x) that first distal surface 24 extends away from base surface 23 of cavity 22. Thus, first distal surface 41 of second electrode element 39 is substantially parallel to first proximal surface 26 of cavity 22, as shown in FIG. 4. Base surface 40 defines a channel 40′ dimensioned to receive a portion of the electrical conductor therein.

FIG. 4 shows a gap between the first distal surfaces 34, 41 of first and second electrode elements 32, 39 and the first distal surface 24 of cavity 22. This gap results from manufacturing tolerances and it will be appreciated by the skilled person that these gaps may vary slight from those shown in the figures.

Second electrode element 39 further comprises an upper surface 45 opposing base surface 40, upper surface 45 being provided with an abutment 46 extending adjacent to and around the whole of the periphery of upper surface 45, but spaced from the outer edge 47 of outer edge upper surface 45.

Second distal surface 42 of second electrode element 39 extends substantially parallel to second distal surface 25 of cavity 22 i.e. second distal surface 42 extends from base surface 40 at the same acute angle (x) as second distal surface 25 extends from cavity base surface 23.

Second distal surface 42 of second electrode element 39 facilitates engagement with and retention within tip portion 18. As first electrode element 32 is located between cavity base surface 23 and second electrode element 39, first electrode element 32 is also retained in place relative to tip portion 18 and movement of first and second electrode elements 32, 39 is restrained in the direction away from the cavity base surface 23 at a transverse angle to the first axis.

Assembly

During assembly of the tip portion 18 of electrosurgical instrument 10, tip portion 18 is located on elongate shaft 12 at distal end region 16. First electrode element 32 is then located within cavity 22 of tip portion 18 such that the first electrode element base surface 33 contacts cavity base surface 23. At this point, neither first nor second distal surfaces 34, 35, contact the first and second distal surfaces 24, 25 of cavity 22. However, first electrode element 32 is held in place due to first proximal surface 36 of first electrode element 32 abutting first proximal surface 26 of cavity 22 and second proximal surface 37 of first electrode element 32 abutting second proximal surface 27 of cavity 22. This serves to locate first electrode element 32 in a spaced relationship from first and second distal surfaces 24, 25 of cavity 22, as shown in FIG. 4.

Electrical conductor 28 is then located within elongate shaft 12 such that distal portion 29 of electrical conductor 28 extends across a portion of channel 38′ of upper surface 38 of first electrode element 32. As can be seen from FIG. 4, channel 38′ is a closed channel and so electrical conductor 28 can only extend a limited distance relative to first electrode element 32.

Second electrical element 39 is then located within cavity 22 of tip portion 18 such that base surface 40 of second electrode element 39 contacts upper surface 38 of first electrode element 32 and a portion of cavity base surface 23 such that channel 38′ is located around at least a portion of electrical conductor 28, thereby securing the distal end of electrical conductor 28 between the first and second electrode elements 32, 39.

There is sufficient clearance between first and second distal surfaces 34, 35 of first electrode element 32 to allow first and second proximal surfaces 36, 37 to form a close fit against first and second proximal surfaces 26, 27 of cavity 22. The angle of first distal surface 31 of second electrode element 39 allows second electrode element 39 to be located in place such that second distal surface 42 abuts second distal surface 25 of cavity 22, thereby retaining both first and second electrode elements 32, 39 in place relative to tip portion 18.

A return brace 48 defining an aperture corresponding to the outer surface is located adjacent the tip portion and the second electrode element, with the aperture aligned with the outer surface of the tip portion. Return brace 46 is located around tip portion 18 as shown in FIG. 1, so as to extend around tip portion 18 and an outer portion of second electrode element 39 i.e. contacting, but not extending beyond, abutment 46 on upper surface 35 of second electrode 39.

Although conventional primary active tip retention typically involves a part of the active tip extending through the adjacent insulator and secured in place with additional riveting, weld plates or other components, for example, active suction tubes, this is not the case with the present design.

The present invention avoids the need for additional rivets, weld plates or other components by relying on the geometry of the various active tip components to securely restrain movement of the electrode in three dimensions i.e. X axis, Y axis and Z axis (see FIG. 2).

Once assembled, no additional retention means are required which allows a lower profile to be achieved at tip portion 18, which is particularly advantageous as the electrosurgical instruments are used in arthroscopic surgery and access is via small incisions (keyhole surgery).

It can be seen from FIG. 4 that retention of active electrode 30 within tip portion 18 is achieved in three different axes:

Active tip retention in the first axis, shown as axis X in FIG. 2, is provided by the extension of electrical conductor 28 into the aperture formed between first and second electrode elements 32, 39, defined by channel 38′ and channel 40′.

Active tip retention in the second axis, shown as axis Y in FIG. 2, is provided by the abutment between second distal surface 25 of cavity 22 and second distal surface 35 and second distal surface 42 of second electrode element 39.

Active tip retention in the third axis, shown as axis Z in FIG. 2, is provided by the abutment between first proximal surface 26 of cavity 22 and the first proximal surface 34 of first electrode element 32.

Thus, the provision of the active tip as a multi-component arrangement facilitates secure tip retention in the X, Y and Z axial directions without the need for additional mechanical components, such as rivets, weld plates or reliance on additional design components such as suction tubes and the like. Instead, the component parts of the active tip can be sequentially welded in place such that the final structure retains the active tip securely in place to provide a securely retained tip portion with reduced profile compared with conventional electrosurgical instruments.

It is to be appreciated that the tip retention of the present invention is applicable to electrode surfaces (active and return) rather than just active tips. For example, the tip retention is also applicable to standard RF bipolar instruments and also to mono-polar devices and devices with more than two active or return elements, for example an instrument with one active tip and two return elements.

1. An electrosurgical instrument comprising:
a hollow elongate shaft extending from the main body along a first axis, the shaft defining an elongate inner open volume which extends from a proximal end region to a distal end region of the shaft;
a tip portion extending from the distal end region of the elongate shaft, the tip portion comprising an electrically insulating material and having an outer surface, the tip portion further defining a cavity therein, the cavity comprising a base surface and a first retention surface extending away from the base surface in a direction towards the distal end region of the shaft at a non-transverse angle to the first axis;
a conductor extending within the shaft from the proximal end region to the distal end region, wherein the proximal end region is suitable for connection to a source of electrosurgical energy; and
an electrode received within the cavity and in electrical communication with the conductor, the electrode comprising a first electrode element and a second electrode element in electrical connection with the first electrode element,
wherein the first and second electrode elements are engaged with one another and the second electrode element abuts and extends adjacent the first retention surface to restrain the movement of the electrode in a direction towards the outer surface of the tip portion and in a direction towards the proximal end region of the shaft.

2. An electrosurgical instrument as claimed in claim 1, wherein the cavity further comprises a second retention surface extending away from the base surface towards the outer surface at an angle transverse to the first axis, the second retention surface facing towards the proximal end region, and wherein the first electrode element abuts the second retention surface and restrains movement of the electrode in a direction towards the distal end region of the shaft.

3. An electrosurgical instrument as claimed in claim 1, wherein the cavity further comprises a third retention surface extending away from the base surface towards the outer surface at an angle transverse to the first axis, the third retention surface facing towards the distal end region, and wherein the first and second electrode elements abut the third retention surface and restrain movement of the electrode in a direction towards the proximal end region of the shaft.

4. An electrosurgical instrument as claimed in claim 1, wherein the conductor extends into the cavity and engages the first and second electrode elements.

5. An electrosurgical instrument as claimed in claim 4, wherein the first and second electrode elements define an aperture dimensioned to receive a portion of the conductor therein and wherein the conductor is located within the aperture restrains movement of the electrode in a direction transverse to the first axis.

6. An electrosurgical instrument as claimed in claim 1, wherein the tip portion cavity comprises a continuous channel extending around the periphery of the tip portion and surrounding the outer surface.

7. An electrosurgical instrument as claimed in claim 1, wherein the first and second electrode elements are welded together within the cavity.

8. An electrosurgical instrument as claimed in claim 6, further provided with a return brace defining an aperture corresponding to the outer surface, wherein the return brace is located adjacent the tip portion and the second electrode element, with the aperture aligned with the outer surface of the tip portion.

9. An electrosurgical instrument as claimed in claim 1, wherein the electrosurgical instrument comprises a bone bur, suction coagulator, bipolar forceps.
